(19) United States
(12) Patent Application Publication
(10) Pub. No.: US 2022/0265589 A1
(43) Pub. Date: Aug. 25, 2022

(54) METHOD OF ADMINISTERING A DUAL THERAPEUTIC AND COSMETIC AGENT

(71) Applicant: Nanomed Skincare, Inc., Cupertino, CA (US)

(72) Inventor: Bai XU, Cupertino, CA (US)

(21) Appl. No.: 17/678,403

(22) Filed: Feb. 23, 2022

Related U.S. Application Data
(60) Provisional application No. 63/152,578, filed on Feb. 23, 2021.

(51) Int. Cl. A61K 31/197; A61K 8/41; A61K 8/368; A61K 8/73; A61K 8/64; A61K 47/12; A61K 47/26; A61K 47/10; A61K 47/22; A61K 47/18; A61Q 19/08; A61Q 19/00; A61Q 19/10; A61K 31/60; A61K 31/728; A61K 38/08

(52) U.S. Cl. CPC A61K 31/197; A61K 8/41; A61K 8/368; A61K 8/735; A61K 8/64; A61K 47/12; A61K 47/26; A61K 47/10; A61K 47/22; A61K 47/18; A61Q 19/08; A61Q 19/007; A61Q 19/10; A61K 31/60; A61K 31/728; A61K 38/08

(57) ABSTRACT

The method of administering a dual therapeutic and cosmetic agent provides for the transdermal application of a single agent which has both therapeutic and cosmetic uses. The composition containing the dual therapeutic and cosmetic agent is selected for therapeutic use, cosmetic use, or a combination thereof. An effective amount of the dual therapeutic and cosmetic agent is transdermally delivered to a recipient in need thereof based on whether therapeutic or cosmetic use is selected. The transdermal delivery may be performed by applying the dual therapeutic and cosmetic agent to skin of the recipient in combination with application of a microneedle array to the skin of the recipient. The application of the dual therapeutic and cosmetic agent to skin of the recipient may be performed before, after or with application of the microneedle array to the skin of the recipient.

### BACKGROUND

### 1. Field

The disclosure of the present patent application relates to transdermal treatments, and particularly to a method of transdermally administering a dual therapeutic and cosmetic agent.

### 2. Description of the Related Art

Transdermal delivery, through microneedle arrays and the like, is commonly used for the delivery of cosmetics, pharmaceuticals and other therapeutic agents. However, there are a number of pharmaceuticals and therapeutic agents which are also known to have cosmetic uses, and vice versa. It would be desirable to be able to use the same type of transdermal delivery that is used with known pharmaceuticals and therapeutic agents for the dual purpose of cosmetic treatment with the same, or similar, agents, or vice versa. Thus, a method of administering a dual therapeutic and cosmetic agent solving the aforementioned problems is desired.

### SUMMARY

The method of administering a dual therapeutic and cosmetic agent provides for the transdermal application of a single agent which has both therapeutic and cosmetic uses. The dual therapeutic and cosmetic agent is selected for therapeutic use, cosmetic use, or a combination thereof. An effective amount of the dual therapeutic and cosmetic agent is transdermally delivered to a recipient in need of the dual therapeutic and cosmetic agent. The effective amount can be different if the desired treatment is a cosmetic treatment or a therapeutic treatment. The transdermal delivery may be performed by applying the dual therapeutic and cosmetic agent to skin of the recipient in combination with application of a microneedle array to the skin of the recipient. The application of the dual therapeutic and cosmetic agent to skin of the recipient may be performed before, after or with application of the microneedle array to the skin of the recipient.

These and other features of the present subject matter will become readily apparent upon further review of the following specification.

### DETAILED DESCRIPTION OF THE PREFERRED EMBODIMENTS

The method of administering a dual therapeutic and cosmetic agent provides for the transdermal application of a single agent which has both therapeutic and cosmetic uses. The dual therapeutic and cosmetic agent is selected for therapeutic use, cosmetic use, or a combination thereof. An effective amount of the dual therapeutic and cosmetic agent is transdermally delivered to a recipient in need of the dual therapeutic and cosmetic agent. The transdermal delivery may be performed by applying the dual therapeutic and cosmetic agent to skin of the recipient in combination with application of a microneedle array to the skin of the recipient. The application of the dual therapeutic and cosmetic agent to skin of the recipient may be performed before, after or with application of the microneedle array to the skin of the recipient. It should be understood that any suitable type of microneedle array may be used. A non-limiting example of such a microneedle array device is shown in U.S. Pat. No. 8,043,250 B2, which is hereby incorporated by reference in its entirety.

As a non-limiting example, tranexamic acid may be delivered to the recipient. Tranexamic acid is used for both skin care as well as for the treatment of melasma. The treatment of melasma using a microneedle array with tranexamic acid is known. For example, such treatment is described in Xu, Yang, et al. “Efficacy of functional microarray of microneedles combined with topical tranexamic acid for melasma: A randomized, self-controlled, split-face study.” Medicine 96.19 (2017), which is hereby incorporated by reference.

In one embodiment, the present methods comprise: treating a topical site of a mammal using a microneedle array, and applying an effective amount of a dual therapeutic and cosmetic agent to the topical site, in any order, to allow the agent to penetrate into the body of the mammal. The microneedle array comprises an array of microstructures. The microstructure can have an aspect ratio of about 5:1, 10:1, 15:1, 20:1 or above.

### Skin Structure

Skin has a biological barrier called stratum corneum in its outer layer. This layer of about 20 microns thick prevents most of the molecules from penetrating through the skin. The layer below the stratum corneum is called viable epidermis. Epidermis is between 50 to 100 micron thick. The viable epidermis layer has no blood vessels and the molecules in this layer can be transported to and from the dermis, a layer under the viable epidermis, which is between 1 to 3 mm thick. There are blood vessels, lymphatics and nerves in dermis layer. To date, a skin patch is only able to deliver drug molecules of less than 500 Da. In addition, these small molecules are typically limited to hydrophobic ones.

### Requirement of Delivery of Drugs, Vaccines and Cosmetic Substances

Successful transdermal delivery of therapeutic drugs, vaccines and cosmetic substances needs a way to transport molecules, especially large molecules through the skin barrier, stratum corneum. The substance can be delivered into the skin in any form acceptable to cosmetic or pharmaceutical requirements.

The microneedle array can be used for effective transdermal delivery of an agent. The microneedle array can comprise a plurality of microstructures formed of a metallic, semi-conductor, glass, ceramic, or polymeric material. In some embodiments, the microneedle array can be microneedles, microknives, or microblades. In some embodiments, the microneedle array comprises microstructures having a nanoscale tip or edge and a microscale body.

Aspect-ratio is defined as the ratio of the depth or height of a structure to its lateral dimension. High-aspect-ratio microstructures typically have an aspect ratio higher than about 5:1 and they may be useful for a variety of purposes. The tips of the microneedle array need to be sharp in order to lower the insertion force, while the body should be high enough to allow it to completely penetrate stratum corneum. A typical size of the needle tip or width of edge on microblades and microknives is smaller than 10 microns, preferably smaller than 5 microns and the height of the microdevices is higher than 20 microns, preferably higher than 50 microns. The aspect ratio of these microdevices can be higher than 10:1 with the size of the tip and edge smaller than 5 microns and the height of microdevices higher than 50 microns. HARMS can thus be used to fabricate microdevices including microneedles, microblades, and microknives for drug delivery through skin or body fluids extraction out of skin. Another example of HARMS is microchannels for microfluidic manipulation and transport. HARMS is typically made by Micro-ElectroMechanical Systems (MEMS) or microfabrication technology that involves a number of thin film deposition, photolithography, etching and electroplating, injection molding, hot embossing, as well as LIGA process.

The microneedle array can contain one or more microneedles. The length of the microneedle is typically in the range of 20-500 microns, sufficient to pierce through the outer skin barrier layer and deliver molecules to viable epidermis or even deeper. The diameter of a single microneedle is typically in the range of 30-300 microns with a sharp tip of less than 10 microns to cause little comfort to the patients while maintaining mechanical integrity. In one embodiment, the needle tip is less than 2 microns and the height of the needle shaft is about 100 microns. The aspect ratio is 50:1.

### Materials and Device Sterilization

The microneedle array can be made of many different materials or their combinations, including metals, ceramics, polymers and glass. Examples of the materials are titanium, stainless steel, nickel, alloy of nickel-iron, silicon, silicon oxide, glass, polymethyl methacrylate (PMMA), polyaryletherketone, nylon, PET, poly(lactic acid), poly(glycolic acid) (PGA), poly(lactic-co-glycolic acid) (PLGA), polycarbonate, and polystyrene. It should have enough mechanical strength to penetrate skin without break and buckle while ensure delivery of drugs, or collect of biological fluids. They can be sterilizable using established protocols (see, for example, moist heat, ethylene oxide or radiation sterilization as stated by ANSI/AAMI/ISO 11134:1993, ANSI/AAMI/ISO 11135:1994 and ANSI/AAMI/ISO 11137:1994).

### The Microchannels

High-aspect-ratio microchannels can be embedded in the microneedle array to allow flexible manipulation of microfluidics and connect microneedles to other functional blocks such as drug reservoirs. Microchannels can be made of many different materials or their combinations, including metals, ceramics, polymers and glass.

### The Microreservoirs

The microneedle array can be connected to a reservoir with drug molecules and their proper formulations in liquid or solid forms. The microreservoir can be made of many different materials or their combinations, including metals, ceramics, polymers and glass. In a preferred embodiment, the microneedles are solid and the microreservoir is connected to the skin surface through microchannels. The applied composition can be dissolved by moisture evaporated from skin and the active substance can diffusion into skin through conduits opened along the interface of skin and microneedles. In another embodiment, the microneedles are hollow and they can be connected with drug reservoir through various microchannels. The reservoir can be made of natural polymers, deformable elastic polymers, metals and ceramics as listed above.

### Method of Use

The microneedle array described herein can be used for transdermal delivery of an agent to treat, prevent, or ameliorate a body condition in need of treatment. The method generally includes treating a skin site of delivery with a microneedle array described herein, and delivery of an agent to the body of a mammal (e.g., a user or patient).

In some embodiments, the agent can be included in the microneedle array as a coating with or without a carrier. In these embodiments, the agent can be delivered with the microdevice being attached to the site of delivery until a desired quantity or duration of delivery is achieved.

In some embodiments, the agent can be separate from the microneedle array. In these embodiments, the skin site chosen for delivery the agent can pre-treated with the microneedle array. The agent can then be applied to the skin site of delivery to allow the agent to penetrate into the body of a user or patient.

The body condition can be a medical condition or a cosmetic condition. Representative medical conditions include, but are not limited to, AIDS, breast cancer, melanoma, liver cancer, lung cancer, blood cancer, pituitary tumors, other cancers, flu, infection, blood disease, cardiac disease, back pain, neck pain, body pain, general pain, arthritis, osteoporosis, headache, depression, smoke, alcoholic, overweight and obesity, menopause, facial hair growth, balding, polycystic ovary syndrome, need of inoculation, need of anesthetics and in particular dermal disease. Representative cosmetic conditions include, but are not limited to, skin aging, skin wrinkle, dark spot, skin discoloration, moisturizing, skin lightening, skin whitening, skin firming, skin lifting, acne, wart, infection, irritation, dry skin and oily skin.

The microneedle arrays are designed as disposable or re-usable devices. In one embodiment, the microneedle arrays are disposable. Depending on whether the microneedle arrays have coating of active substances on them or not, there are three categories of applications in the delivery of drugs, cosmetic substances and vaccines.

For delivery of an agent, in one embodiment, the microneedle array can be used to perforate or scratch stratum corneum. They are then removed immediately and a skin patch with active substance is applied to the microdevice treated area right away. The skin patch will stay on the skin for a pre-defined period, providing sustainable controlled release of active substances.

Another embodiment is to store the active agents in the substrate and rely on passive diffusion when the microneedle array is in touch with skin.

In yet a further embodiment, one can pre-coat microneedle shafts with a formulation that contains active substances. The coated microneedles are applied to the skin and stay on the skin for the entire period of treatment. The rate of through skin transport can be measured using in vitro or in vivo methods known in the art.

### Example 1

As a non-limiting example of a tranexamic acid-based treatment, the dual therapeutic and cosmetic agent may have the following composition: 5 vol % tranexamic acid, 2 vol % trehalose, 2 vol % 1,3-propanediol, 0.25 vol % xanthan gum, 90.05 vol % water and 0.7 vol % a compound formed from 39 vol % 1,2-diol, 36 vol % 1,3-propanediol, 13 vol % octyl hydroxamic acid, and 12 vol % ethylhexylglycerol.

### Example 2

As a non-liming example of a tranexamic acid-based treatment, the dual therapeutic and cosmetic agent may have the following composition: 5 vol % tranexamic acid, 2 vol % trehalose, 2 vol % 1,3-propanediol, 0.3 vol % sodium hyaluronate, 90.0 vol % water and 0.7 vol % a compound formed from 39 vol % 1,2-diol, 36 vol % 1,3-propanediol, 13 vol % octyl hydroxamic acid, and 12 vol % ethylhexylglycerol.

### Example 3

As a non-liming example, the dual therapeutic and cosmetic agent may be a salicylic acid-based acne formula having the following composition: 40 vol % glycerol, 30 vol % 1,3-butanediol, 29.6 vol % 1,3-propanediol, and 0.4 vol % salicylic acid.

### Example 4

As a non-liming example, the dual therapeutic and cosmetic agent may be a salicylic acid-based acne formula having the following composition: 99.1 vol % water, 0.5 vol % sodium hyaluronate, and 0.4 vol % salicylic acid.

### Example 5

As a non-liming example, the dual therapeutic and cosmetic agent may be a wrinkle eliminating formula having the following composition: 97.8 vol % water, 0.05 vol % acetyl hexapeptide-8, 0.5 vol % sodium hyaluronate, 0.6 vol % trehalose, 0.25 vol % allantoin, and 0.8 vol % a compound formed from 39 vol % 1,2-diol, 36 vol % 1,3-propanediol, 13 vol % octyl hydroxamic acid, and 12 vol % ethylhexylglycerol.

### Example 6

As a non-liming example, the dual therapeutic and cosmetic agent may be a hair loss prevention formula having the following composition: 3 vol % cetearyl alcohol-25 (e.g., TEGINACID® C), 0.1 vol % dihydrosphingosine (e.g., SPHINGONY®), 50.0 vol % anhydrous ethanol, 0.1 vol % essence, a sufficient quantity of preservatives, a sufficient quantity of lactic acid to produce a pH of 5.5, and a remainder of water.

### Example 7

As a non-liming example, the dual therapeutic and cosmetic agent may be a hair loss prevention formula having the following composition: 2.0 vol % a solubilizer (e.g., TEGO® Solve 61), 2.0 vol % a hair cleaning agent (e.g., RHEANCE® One), 0.1 vol % dihydrosphingosine (e.g., SPHINGONY®), 3.0 vol % glycerin, a sufficient quantity of preservatives, a sufficient quantity of lactic acid to produce a pH of 6.0, and a remainder of water.

### Example 8

As a non-liming example, the dual therapeutic and cosmetic agent may be a liquid foundation formula having the following composition: 21.41 vol % glycerol, 1.0 vol % sodium hyaluronate, 11.52 vol % titanium dioxide, 5.76 vol % pearl powder, 0.45 vol % xanthan gum, 59.35 vol % water, and 0.5 vol % a compound formed from 39 vol % 1,2-diol, 36 vol % 1,3-propanediol, 13 vol % octyl hydroxamic acid, and 12 vol % ethylhexylglycerol.

### Example 9

As a non-liming example, the dual therapeutic and cosmetic agent may be a water locking cream formula having the following composition: 5.0 vol % olive oil, 5.0 vol % almond oil, 2.5 vol % squalene, 2.5 vol % glycerin, 1.0 vol % 1,2-diol, 1.0 vol % 1,3-propanediol, 1.70 vol % an emulsifier (e.g., Simulgel EG manufactured by Seppie), 80.8 vol % water, and 0.5 vol % a compound formed from 39 vol % 1,2-diol, 36 vol % 1,3-propanediol, 13 vol % octyl hydroxamic acid, and 12 vol % ethylhexylglycerol.

### Example 10

As a non-liming example, the dual therapeutic and cosmetic agent may be an amino acid facial cleanser formula having the following composition: 1.0 vol % pearl slice, 1.0 vol % 12-hydroxylauric acid (an emulsifying, cleaning and foaming agent), 0.5 vol % 18-hydroxystearic acid (an emulsifying, stabilizing, suspending and skin sensation improving agent), 0.96 vol % citric acid (an exfoliating, pH conditioning and antibacterial agent), 6.0 vol % coconut propyl betaine (an antistatic, cleaning and foaming agent), 5.0 vol % PEG-7 glycerol coconate (a solvent, emulsifier and surfactant), 20 vol % glycerin, 15 vol % sodium coconoacyl glycine, 5.0 vol % an amino acid foaming agent, 0.80 vol % carbomer, 0.50 vol % triethanolamine, and 0.8 vol % a compound formed from 39 vol % 1,2-diol, 36 vol % 1,3-propanediol, 13 vol % octyl hydroxamic acid, and 12 vol % ethylhexylglycerol. In the above, the pearl slice, 12-hydroxylauric acid and 18-hydroxystearic acid are part of an oil phase of the overall composition, and the remainder are part of a water phase.

It is to be understood that the method of administering a dual therapeutic and cosmetic agent is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. A method of administering a dual therapeutic and cosmetic agent to a recipient in need thereof, comprising the steps of:
a recipient in need selecting between a cosmetic treatment and a therapeutic treatment with a dual therapeutic and cosmetic agent; and
transdermally delivering to the recipient in need a composition comprising an amount of the dual therapeutic and cosmetic agent effective for the selected cosmetic or therapeutic treatment.

2. The method of administering a dual therapeutic and cosmetic agent as recited in claim 1, wherein the step of transdermally delivering to the recipient in need the composition comprises applying the composition to skin of the recipient in combination with application of a microneedle array to the skin of the recipient.

3. The method of administering a dual therapeutic and cosmetic agent as recited in claim 2, wherein the application of the composition to the skin of the recipient is performed prior to the application of the microneedle array to the skin of the recipient.

4. The method of administering a dual therapeutic and cosmetic agent as recited in claim 2, wherein the application of the composition to the skin of the recipient is performed following the application of the microneedle array to the skin of the recipient.

5. The method of administering a dual therapeutic and cosmetic agent as recited in claim 2, wherein the application of the composition to the skin of the recipient is performed simultaneously with the application of the microneedle array to the skin of the recipient.

6. The method of administering a dual therapeutic and cosmetic agent as recited in claim 1, wherein the dual therapeutic and cosmetic agent comprises up to 10 vol % tranexamic acid.

7. The method of administering a dual therapeutic and cosmetic agent as recited in claim 1, wherein the composition further comprises one or more therapeutically or cosmetically acceptable excipients.

8. The method of administering a dual therapeutic and cosmetic agent as recited in claim 7, wherein the one or more therapeutically or cosmetically acceptable excipients are selected from the group consisting of trehalose, 1,3-propanediol, xanthan gum, 1,2-diol, sodium hyaluronate, water, octyl hydroxamic acid, ethylhexylglycerol, glycerol, 1,3-butanediol, allantoin, acetyl hexapeptide-8, cetearyl alcohol, dihydrosphingosine, glycerine, anhydrous ethanol, essence, preservatives, lactic acid, a pH modifier, a buffer, a solubilizer, a hair cleaning agent, titanium dioxide, pearl powder, a coloring agent, olive oil, almond oil, squalene, an emulsifier, pearl slice, hydroxylauric acid, hydroxystearic acid, citric acid, coconut propyl betaine, glycerol coconate, sodium coconoacyl glycine, an amino acid foaming agent, carbomer, triethanolamine, and combinations thereof.

9. The method of administering a dual therapeutic and cosmetic agent as recited in claim 1, wherein the dual therapeutic and cosmetic agent comprises salicylic acid.

10. The method of administering a dual therapeutic and cosmetic agent as recited in claim 1, wherein the dual therapeutic and cosmetic agent comprises hyaluronic acid.

11. The method of administering a dual therapeutic and cosmetic agent as recited in claim 1, wherein the dual therapeutic and cosmetic agent comprises a hexapeptide.

12. The method of administering a dual therapeutic and cosmetic agent as recited in claim 1, wherein the composition contains a sufficient quantity of lactic acid to produce a pH of about 5.5-6.0.
